(19) United States
(12) Patent Application Publication
(10) Pub. No.: US 2026/0157680 A1
(43) Pub. Date: Jun. 11, 2026

(54) IMPLANTABLE SENSOR STRAND

(71) Applicant: Phantom Neuro Inc., Austin, TX (US)

(72) Inventors: Connor Guthman Glass, Austin, TX (US); Michael Faltys, Austin, TX (US)

(21) Appl. No.: 19/181,604

(22) Filed: Apr. 17, 2025

Related U.S. Application Data
(60) Provisional application No. 63/635,290, filed on Apr. 17, 2024.

(51) Int. Cl. A61B 5/389; A61B 5/00; A61B 5/296

(52) U.S. Cl. CPC A61B 5/389; A61B 5/296; A61B 5/6846; A61B 2562/046

(57) ABSTRACT

A patient implantable system for subdermal collection of patient data includes a sensor strand including a plurality of sensors housed within a tubular body and an electronics suite. The sensors include electrodes configured to detect electromyography (EMG) and positional signals. The sensor strand is mechanically configurable to have a first shape during insertion and a second shape during operation, the second shape arranging the plurality of sensors in two or more rows. The electronics suite includes a housing, and a processor receiving signals from the sensors that are processed to deliver a physiologically assisting operation.

### TECHNICAL FIELD

The present disclosure generally relates to subdermal implants and more specifically to implantable sensor strands.

### BACKGROUND

Intracorporeal implantable devices especially implantable sensor assemblies have been used to collect patient data to generate signals for assisting devices that compensate for a variety of physical conditions including mobility impairments resulting from aging and/or physical disabilities. The output of the assisting devices is based on data processing and can be improved by increasing the amount of collected patient data by using a larger number of sensors. Implantable hardware including a larger number of sensors placed in the body can increase the risks associated with implantation procedures, infection, discomfort, and recovery.

### SUMMARY

Implementations of the present disclosure are directed to subdermal implants. More particularly, implementations of the present disclosure are directed to implantable sensor strands forming open end bracelets with adjustable shapes and sizes.

In some implementations, a patient implantable system for subdermal collection of patient data includes: a sensor strand including a plurality of sensors housed within a tubular body, the plurality of sensors including electrodes configured to detect electromyography (EMG) and positional signals, wherein the sensor strand is mechanically configurable to have a first shape during insertion and a second shape during operation, the second shape arranging the plurality of sensors in two or more rows, and an electronics suite including a housing, and a processor receiving signals from the plurality of sensors.

The foregoing and other implementations can each optionally include one or more of the following features, alone or in combination. In particular, implementations can include but are not limited to the following features:

In some aspects, combinable with any of the previous aspects, the sensor strand staggers, flexes, or bends in a plurality of directions. The patient implantable system further includes a draw string of an adjustable length controlling the second shape of the sensor strand. The second shape defines a sensor array bracelet. The sensor array bracelet is mechanically configurable to have a variable radius. The sensor array bracelet includes an open-end bracelet or a coiled bracelet. The sensor array bracelet includes circumferentially distanced ends or longitudinally distanced ends. The electronic suite is coupled to a prosthetic device. The electronic suite includes a non-rechargeable battery or a rechargeable battery. The rechargeable battery is coupled to a battery charging coil. The battery is hermetically or non-hermetically sealed. The electronic suite is attached to an end of the sensor strand or a middle portion of the sensor strand. The electronics suite is attached to a reference electrode. The electronic suite includes an antenna for transmitting the signals received from the plurality of sensors. The housing of the electronics suite includes a disc shape or a tubular shape with smooth edges. The patient implantable system includes a plurality of feedthroughs transmitting the patient data from the sensory strand through the housing of the electronics suite by maintaining an hermeticity of the housing. The sensor strand includes up to 32 electrodes. The sensor strand is formed of a flexible material coupled to a stylet shaping the sensor strand for insertion.

Other implementations of the aspect include corresponding systems, apparatus, and computer programs, configured to perform the actions of the methods, encoded on computer storage devices.

The present disclosure also provides a computer-readable storage medium coupled to one or more processors and having instructions stored thereon which, when executed by the one or more processors, cause the one or more processors to perform operations in accordance with implementations of the methods provided herein.

In some implementations, a patient implantable system for subdermal collection of patient data includes: a sensor strand including a plurality of sensors, wherein the sensor strand is mechanically configurable to have a first shape during insertion and a second shape during operation, the second shape arranging the plurality of sensors in two or more rows, and an electronics suite including a housing, and an electronic circuit disposed within the housing, the electronic circuit receiving signals from the plurality of sensors, the housing including a polymeric encapsulation layer surrounding the electronic circuit, providing a non-hermetic seal that protects the electronic circuit from bodily fluids.

In some aspects, combinable with any of the previous aspects, the polymeric encapsulation layer includes at least one of polyurethane, silicone, or polyethylene. The encapsulation layer provides environmental resistance during a temporary implantation, and provides controlled access to the electronic circuit for replacement or revision. The electronic circuit includes a coating applied directly to the electronic circuit prior to encapsulation. The housing is flexible, conforming to anatomical structures during or after implantation. The sensors are configured for neuromodulation or cardiac monitoring. The polymeric encapsulation is applied using a dip-coating, spray-coating, or overmolding process.

In some implementations, a method of using a patient implantable system for collecting internal patient data includes: inserting a stylet within a sensor strand including a plurality of sensors housed within a tubular body, the plurality of sensors including electrodes configured to detect electromyography (EMG) and positional signals, wherein the sensor strand has a first shape during insertion, positioning the sensor strand between tissue layers, and actuating a strand shape controller for the sensor strand to form and maintain a second shape during operation.

In some aspects, combinable with any of the previous aspects, the stylet is removed after forming the second shape.

In some implementations, a modular insertion system includes: a flexible conduit configured for implantation or subcutaneous positioning between tissue planes of a limb, and a removable stylet positioned within the conduit, the stylet having a stiffness that facilitates insertion of the conduit between anatomical tissue planes.

In some aspects, combinable with any of the previous aspects, the removable stylet is removed after placement of the flexible conduit, wherein the flexible conduit assumes a neutral or more conformable shape governed by a flexibility of a conduit material. The removable stylet is replaced with a second stylet including a greater flexibility than the removable stylet, the second stylet imparting a smooth curvature following contours of the limb. The removable stylet includes a pre-formed curvature and a stiffness level adapted to conform to an anatomical structure and an appendage size. The removable stylet is composed of a shape-memory material. The shape-memory material is insertable in a substantially straight configuration. The shape-memory material in response to exposure to body temperature or physiological conditions, assumes a curved or coiled geometry to conform to a surrounding anatomical structure.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

It is appreciated that methods in accordance with the present disclosure can include any combination of the aspects and features described herein. That is, methods in accordance with the present disclosure are not limited to the combinations of aspects and features specifically described herein, but also include any combination of the aspects and features provided.

Implementations described in the present disclosure, provide multiple technical advantages over traditional implantable sensors. The described technology provides implantable sensor strands forming open end bracelets with adjustable shapes and sizes. As an advantage, the described implantable hardware includes a sensor strand designed to minimize the overall volume of the hardware material placed in the body and simultaneously maximizes the covered sensor detection area. The minimized overall volume of the hardware material placed in the body reduces the risks associated with implantation procedures, infection, discomfort, and recovery time. Furthermore, the maximized covered sensor detection area increases the accuracy and applicability of the collected sensor data. Another advantage of the described technology is that the described sensor strand has a configuration that can be adjusted to optimize personalized placement, matching the anatomical features of each patient. The described sensor strand can be shaped as a squiggly bracelet with an adjustable length and diameter that can be varied by modifying the loop angles and amplitudes using a draw string. Another advantage of the described technology is that the tubular configuration of the sensor strand and the smooth rounded edges of the electronic suite casing increase implant biocompatibility, minimizing tissue scarring due to implant insertion and optimizing recovery.

The details of one or more implementations of the subject matter of the specification are set forth in the accompanying drawings and the description below. Other features, aspects, and advantages of the subject matter can become apparent from the description, the drawings, and the claims.

### DESCRIPTION OF THE DRAWINGS

The accompanying drawings, which are incorporated in and constitute a part of this specification, show particular aspects of the subject matter disclosed herein and, together with the description, help explain some of the principles associated with the disclosed implementations. In the drawings,

FIG. 1 is a block diagram of an example system for using an implantable system, in accordance with some example implementations;

FIG. 2 is a block diagram of an example system for using an implantable system, in accordance with some example implementations;

FIG. 3A depicts a schematic diagram illustrating an example system including multiple patient implantable systems, in accordance with some example implementations;

FIG. 3B shows a schematic diagram of an example implantable battery-powered implantable sensor strand, in accordance with some example implementations.

FIG. 4A depicts a schematic diagram illustrating an example patient implantable system, in accordance with some example implementations;

FIG. 4B depicts a schematic diagram illustrating a cross section of the example patient implantable system of FIG. 4A, in accordance with some example implementations;

FIG. 4C depicts a schematic diagram illustrating an example electronics suite of the example patient implantable system of FIG. 4A, in accordance with some example implementations;

FIG. 5A depicts a schematic diagram illustrating another example patient implantable system, in accordance with some example implementations;

FIG. 5B depicts a schematic diagram illustrating a longitudinal section of the example sensor strand in a straight configuration of the example patient implantable system of FIG. 5A, in accordance with some example implementations;

FIG. 5C depicts a schematic diagram illustrating the example patient implantable system of FIG. 5B during insertion, in accordance with some example implementations;

FIG. 5D depicts a schematic diagram illustrating a longitudinal section of the example sensor strand in a bent configuration of the example patient implantable system of FIG. 5A, in accordance with some example implementations;

FIG. 5E depicts a schematic diagram illustrating the example patient implantable system of FIG. 5D during operation, in accordance with some example implementations;

FIG. 6A depicts a schematic diagram illustrating another example patient implantable system, in accordance with some example implementations;

FIG. 6B depicts a schematic diagram illustrating a longitudinal section of the example sensor strand of the example patient implantable system of FIG. 6A, in accordance with some example implementations;

FIG. 6C depicts a schematic diagram illustrating another longitudinal section of the example sensor strand of the example patient implantable system of FIG. 6A, in accordance with some example implementations;

FIG. 7A depicts a schematic diagram illustrating an example patient implantable system, in accordance with some example implementations;

FIG. 7B depicts a schematic diagram illustrating another example patient implantable system, in accordance with some example implementations;

FIG. 7C depicts a schematic diagram illustrating another example patient implantable system, in accordance with some example implementations;

FIG. 7D depicts a schematic diagram illustrating another example patient implantable system, in accordance with some example implementations;

FIG. 8 depicts a schematic flow diagram illustrating an example process of inserting and using an example patient implantable system, in accordance with some example implementations; and

FIG. 9 depicts a block diagram illustrating a computing system, in accordance with some example implementations.

When practical, like labels are used to refer to same or similar items in the drawings.

### DETAILED DESCRIPTION

Implementations of the present disclosure are directed to subdermal implants. More particularly, implementations of the present disclosure are directed to implants including sensor strands for subdermal collection of patient data. The described implementations provide sensor strands including multiple sensors housed within a tubular body configured to detect electromyography (EMG) and positional signals. The described sensor strand is mechanically configurable to have a first shape during insertion and a second shape during operation, the second shape arranging the sensors in two or more rows. Each of the implants includes an electronics suite including a housing and a processor receiving signals from the sensors.

The evolution of medical technology has continuously aimed at making medical systems more efficient in terms of power consumption and less invasive. Traditional implants including sensor arrays are shaped as flat bands presenting a wide surface enabling distribution of sensors across multiple rows. The size of the sensors and the width of the support band dictate the number of sensor rows that can be formed, such that according to the traditional implant configuration, wider bands are used as substrates for larger numbers of sensor rows. With an average width band, the post-operative recovery time of a patient can last several weeks to a month. For example, using traditional implantable electromyography devices, healthcare providers regularly balance the risks involved by inserting wider bands (requiring larger incisions and increasing the risk of complications) to the need to record greater spatial data using a greater number of multiple rows of sensors. The traditional band supported sensor arrays are limited in providing options to reduce hardware size while simultaneously maximizing surface area covered with a respective sensor array. Additionally, in conventional patient implantable systems, circuits are typically hermetically sealed to ensure long-term reliability and protection from bodily fluids. Hermetic sealing involves encasing electronic components in a hermetically sealed housing, using materials like metal and glass, which are impermeable to gases and liquids. Hermetic seals are effective in maintaining the integrity and functionality of the electronics over extended periods. For example, hermetic sealing ensures that electronic components remain unaffected by the corrosive effects of bodily fluids. The durability of hermetically sealed systems can minimize revisions and maintenance checks. The stability provided by hermetic sealing contributes to consistent performance of the implantable system. As a drawback, the materials and design required for hermetic sealing can lead to more invasive surgical procedures. The complexity of creating a hermetically sealed systems adds to the manufacturing time and effort to produce hermetically sealed implants.

The implementations described in the present disclosure, provide multiple technical advantages. In particular, the described implant includes a sensor array housed within a tubular body that overcomes the limitations of traditional implants including band supported sensor arrays. An advantage of the implementations described in the present disclosure is that the described sensor strand minimizes the overall volume of the hardware material placed in the body that simultaneously maximizes the covered sensor detection area. The minimized overall volume of the hardware material placed in the body reduces the recovery time to approximately one week. The minimized overall volume of the hardware material placed in the body also reduces the risks associated with implantation procedures, infection, and discomfort. The advantages stemming from the minimized overall volume of the hardware material are further increased by the geometry of the sensor strand and of the electronic suite, positively impacting the implant biocompatibility, minimizing tissue scarring due to implant insertion, and optimizing the recovery process. Furthermore, the maximized covered sensor detection area increases the accuracy and applicability of the collected sensor data. Another advantage of the described technology is that the described sensor strand has a configuration that can be adjusted to optimize personalized placement, matching the anatomical features of each patient. The described sensor strand can be shaped as a squiggly bracelet with an adjustable length and diameter that can be varied by modifying the loop angles and amplitudes using a draw string. The described implant system ensures long-term stability, safety, and biocompatibility while enabling a smaller, more efficient implant design. Furthermore, even though the implantable hardware is described with reference to controlling a prosthetic device, the described implants are compatible with a variety of sensor types providing great versatility for different healthcare treatments that require continuous patient data monitoring. For example, the described implants can be integrated in the following healthcare protocols: cardiac care (e.g., configuring the implantable sensors to monitor heart rate, rhythm, and other cardiac parameters, helping in the management of conditions like arrhythmias, heart failure, and post-surgical recover), diabetes management (e.g., configuring the implantable sensors to track blood sugar levels in real-time, allowing for better management of diabetes and timely adjustments to insulin therapy), neurological disorders (e.g., configuring the implantable sensors to monitor brain activity and detect seizures in patients with epilepsy, providing data that can help in adjusting medications and treatment plans), chronic respiratory diseases (e.g., configuring the implantable sensors to measure lung function and oxygen levels, aiding in the management of conditions like chronic obstructive pulmonary disease and asthma), post-surgical monitoring (e.g., configuring the implantable sensors to track vital signs and detect complications early in patients recovering from surgery, ensuring timely interventions), cancer treatment: sensors can monitor tumor growth and response to treatment, providing valuable data for adjusting therapies and improving outcomes), and renal care (e.g., configuring the implantable sensors to monitor kidney function and detect early signs of complications in patients with chronic kidney disease or those undergoing dialysis).

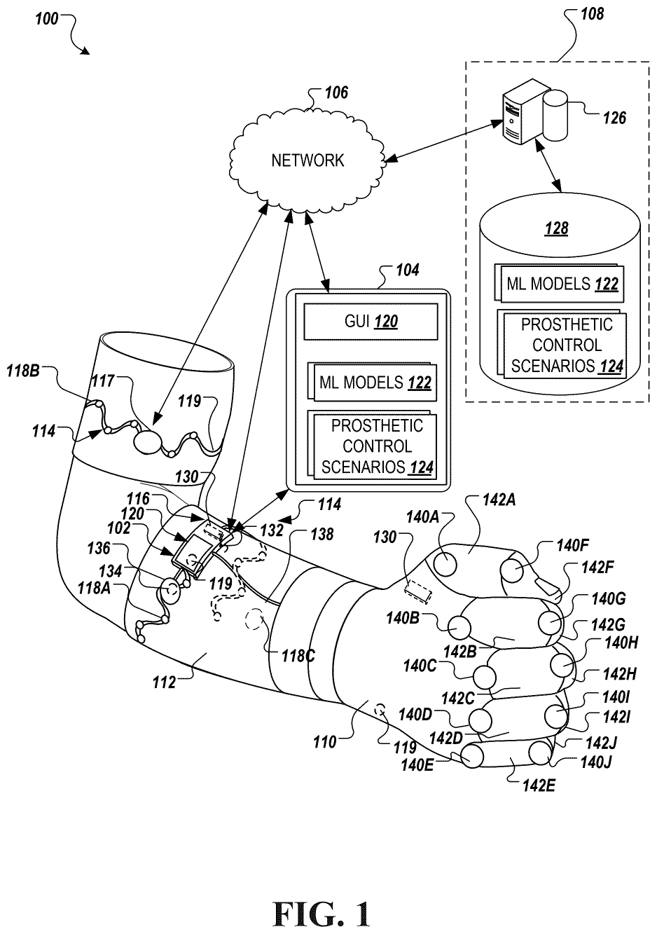

Another advantage of the described technology is that the patient implantable system includes a non-hermetic seal, which utilizes polymers for circuit protection rather than traditional hermetic sealing methods. The polymers offer a flexible, lightweight, and cost-effective alternative to traditional hermetic materials. By utilizing polymers, the implantable system can be designed to be less invasive, while providing sufficient protection to the electronic components. Polymers, while protective, may not offer the same level of long-term durability as hermetically sealed systems. The performance of polymer-based systems can be affected by changes in environmental conditions within the body. The non-hermetic seal facilitates a reduction in size that minimizes invasiveness, being compatible with technical revisions (e.g., electronic component, such as battery, replacement). The intricacies, advantages, and other technical considerations of implementing non-hermetic versions of patient implantable systems as well as other advantages of the implantable systems are described with reference to FIGS. 1-9.

### Example System for Using Implantable Systems

FIG. 1 is a block diagram illustrating an example system 100 for controlling prosthetic devices in accordance with some implementations of the present disclosure. Specifically, the illustrated example system 100 includes or is communicably coupled with a communication controller 102, a user device 104, a network 106, a server system 108, a prosthetic device 110, a support system 112, an implant system 114, and a power supply system 116.

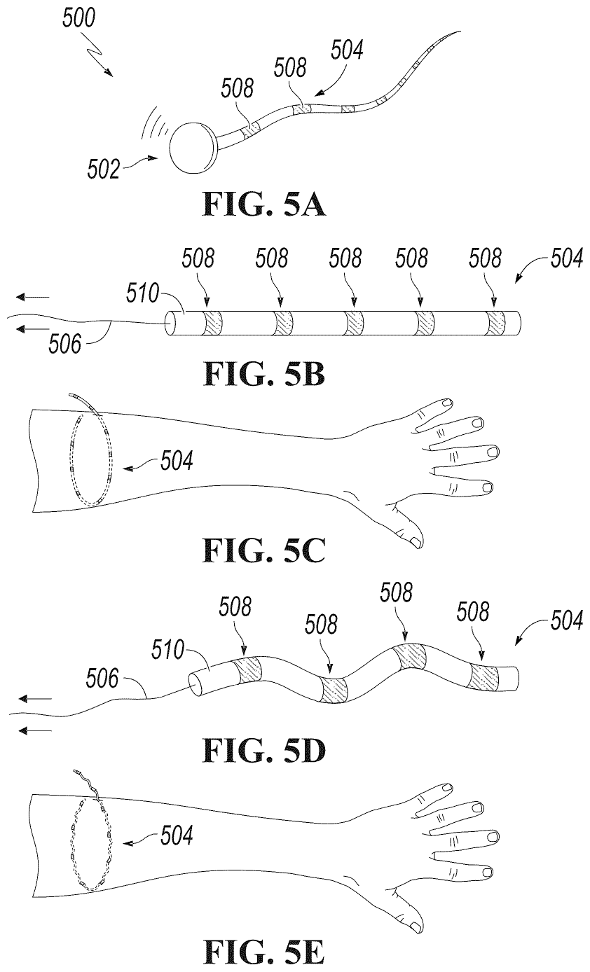

The communication controller 102 includes a hardware processor 126, a memory 128 for storing instructions, a wireless communications device (as described in detail with reference to FIG. 5), one or more IMUs 121, and a user interface 120. The hardware processor 126 facilitates training and execution of machine learning algorithms for prosthetic device control, according to the system configuration. The memory 128 stores training data, machine learning models, and system configuration including instructions for prosthetic device control. The wireless communications device for the implant includes near-field communication (NFC), mid-field communication (proprietary RF), and far-field RF (Wi-Fi) for communication with remote server systems.

During operation, the communication controller 102 receives wireless signals (including EMG signals) from one or more of sensors 118A, 118B, 118C of one or more implant systems 114. The communication controller 102 receives positional data from integrated IMUs 121. The communication controller 102 processes the received wireless signals to generate a prosthetic control signal that is sent to the prosthetic device to actuate one or more joints of the prosthetic device. The communication controller 102 includes a user interface 120 to facilitate direct interaction with the communication controller 102, without the use of a user device. Wireless IMUs 121 are placed on a limb or body part to provide contextual data in addition to EMG signals to provide signals to the communication controller 102. The wireless IMUs 121 are placed on the prosthetic device or exoskeleton to detect the spatial position to enhance the prosthetic control algorithms (e.g. wrist rotation). The communication controller 102 processes the received wireless signals and generates output signals that the communication controller 102 can transmit to the user device 104 (e.g., in a training mode) or to the prosthetic device 110 (as control signals, in an operational usage mode).

The user device 104 can be communicatively coupled to the communication controller 102 and the server system 108, through the network 106. In some implementations, the network 106 can support a short-range communication network, managed by the communication controller 102, and a wide range communication network, accessible through the user device 104. The short-range range communication network can include radio frequency (RF) based network (e.g., using a 2.4 GHz RF link), Bluetooth, Wi-Fi, and/or other such transceiver modules (as described in detail with reference to FIGS. 13A and 13B). The wide range communication network can include a wireless local-area network (WLAN), a local area network (LAN), a wide area network (WAN), the Internet, a cellular network, a telephone network or an appropriate combination thereof connecting any number of user devices 104 and server systems 108 (e.g., during a training mode of the communication controller 102 for controlling the prosthetic device 110). Data exchanged over the network 106, is transferred using any number of network layer protocols, such as Internet Protocol (IP), Multiprotocol Label Switching (MPLS), Asynchronous Transfer Mode (ATM), and Frame Relay. Furthermore, in implementations where the network 106 represents a combination of multiple sub-networks, different network layer protocols are used at each of the underlying sub-networks. In some implementations, the network 106 represents one or more interconnected internetworks, such as the public Internet.

The user device 104 can be any computing device operable to connect to or communicate in the network(s) 108 using a wireline or wireless connection. In general, the user device 104 includes an electronic computer device operable to receive, transmit, process, and store any appropriate data associated with the system 100 of FIG. 1, such as data received from the communication controller 102 and the server system 108, as described with reference to FIG. 15. The user device 104 is generally intended to encompass any client computing device such as a laptop/notebook computer, wireless data port, smart phone, personal data assistant (PDA), tablet computing device, one or more processors within these devices, or any other suitable processing device. The user device 104 includes interface(s), processor(s), memory, and a graphical user interface 120. The user device 104 can include one or more applications. The user device 104 can be configured to execute an application that allows the user device 104 to request and view content on the user device (e.g., initiate a training mode to train the communication controller 102 to control the prosthetic device 110). For example, the user device 104 can include a computer that includes an input device, such as a keypad, touch screen, or other device that can accept user information, and an output device that conveys information associated with a machine learning (ML) model 122 of the server system 108, or the user device itself, including digital data, visual information, or a GUI 120, respectively. The GUI 120 can interface with at least a portion of the system 100 for any suitable purpose, including generating a visual representation of the prosthetic control scenarios 124. Generally, the GUI 120 provides the user device 104 with an efficient and user-friendly presentation of training data provided by or communicated within the system 100 during a training mode. The GUI 120 can include multiple customizable frames or views having interactive fields, pull-down lists, and buttons operated by the user. The GUI 120 can include any suitable graphical user interface, such as a combination of a generic web browser, intelligent engine, and command line interface (CLI) that processes information and efficiently presents the results to the user visually. There can be any number of user devices associated with, or external to, the system 100. Additionally, there can also be one or more additional user devices external to the illustrated portion of system 100 that are capable of interacting with the system 100 using the network(s) 108. Further, the term “client,” “user device,” and “user” can be used interchangeably as appropriate without departing from the scope of the disclosure. Moreover, while user device can be described in terms of being used by a single user, the disclosure contemplates that many users can use one computer, or that one user can use multiple computers.

In the example of FIG. 1, the server system 108 is intended to represent various forms of servers including, but not limited to a web server, an application server, a proxy server, a network server, and/or a server pool. In general, server systems 108 accept requests for application services and provides such services to any number of user devices 104 (e.g., the user device 104 over the network 106). In accordance with implementations of the present disclosure, and as noted above, the server system 108 can host a solution environment that can be a cloud environment providing software applications, systems, and services that can be consumed by customers as a service. In some instances, the server system 108 can support training of the communication controller 102 to control the prosthetic device 110. The server system 108 includes a processor 126, a memory 128, and an interface. The processor 126 included in the server system 108 or the user device 104 can be a central processing unit (CPU), an application specific integrated circuit (ASIC), a field-programmable gate array (FPGA), or another suitable component. Generally, the processor 126 included in the server system 108 (or the user device 104) executes instructions and manipulates data to perform the operations of the server system 108 or the user device 104, respectively. Specifically, the processor 126 executes the functionality required to process/send requests to perform training operations. The processor 126 can be a central processing unit (CPU), a blade, an application specific integrated circuit (ASIC), a field-programmable gate array (FPGA), or another suitable component. As used in the present disclosure, the term “computer” is intended to encompass any suitable processing device. For example, although FIG. 1 illustrates a single server system 108 and a single user device 104, the system 100 can be implemented using a single, stand-alone computing device, two or more servers 108, or multiple user devices. The server system 108 and the user device 104 can include any computer or processing device such as, for example, a blade server, general-purpose personal computer (PC), Mac®, workstation, UNIX-based workstation, or any other suitable device. In other words, the present disclosure contemplates computers other than general purpose computers, as well as computers without conventional operating systems. Further, the server system 108 and the user device 104 can be adapted to execute any operating system or runtime environment, including Linux, UNIX, Windows, Mac OS®, Java™, Android™, iOS, BSD (Berkeley Software Distribution) or any other suitable operating system. According to one implementation, the server system 108 can also include or be communicably coupled with an e-mail server, a Web server, a caching server, a streaming data server, and/or another suitable server.

The memory 128 can include ML model 122 and prosthetic control scenarios 124 used for training and updating the training of the communication controller 102 to control the prosthetic device 110. The memory 128 can include any type of memory or database module and can take the form of volatile and/or non-volatile memory including, without limitation, magnetic media, optical media, random access memory (RAM), read-only memory (ROM), removable media, or any other suitable local or remote memory component. The memory 128 can store various objects or data, including caches, classes, frameworks, applications, backup data, application objects, jobs, web pages, web page templates, database tables, database queries, repositories storing application data and/or dynamic information, and any other appropriate information including any parameters, variables, algorithms, instructions, rules, constraints, or references thereto associated with the purposes of the server system 108, the communication controller 102, and the user device 104, respectively.

Regardless of the particular implementation, “software” can include computer-readable instructions, firmware, wired and/or programmed hardware, or any combination thereof on a tangible medium (transitory or non-transitory, as appropriate) operable when executed to perform at least the processes and operations described herein. Indeed, each software component can be fully or partially written or described in any appropriate computer language including C, C++, Java™, JavaScript®, Visual Basic, assembler, Perl®, ABAP (Advanced Business Application Programming), ABAP OO (Object Oriented), any suitable version of 4GL, as well as others.

In some implementations, the communication controller 102 is powered by the power supply system 116. For example, the communication controller 102 is powered by an external battery pack 130 connected to a power connector 132 of the communication controller 102. The external battery pack 130 can include a primary battery comprising a prismatic cell with built-in protection circuitry and a secondary battery comprising a lithium-ion battery. The communication controller 102 supplies power, such as transcutaneous radio frequency (RF) power, to an electronics suite 117 and/or the sensors 118A, 118B, 118C of the implant system 114. For example, the communication controller 102 supplies power, through the power coil 134, to the implant(s) 118A, 118C housed within a sensor strand 119 having a tubular body that can be attached to or completely detached from (a bottom layer of) the support system 112 (prosthesis socket). For example, the implant system 114 can be implanted in a pocket formed within the subcutaneous or sub-adipose or subfascial anatomical planes of a subject. In some implementation, the communication controller 102 can be connected to and powered by the external battery pack 130 included in the prosthetic device 110. The communication controller 102 can connect to the prosthetic device 110 and can route power from the battery pack 130 to the prosthetic device 110, supplying the prosthetic device 110 and other components of the example system 100 with the necessary power for operation.

The communication controller 102 can also supply power to remote sensors 118B (positioned at a distance greater than 2 cm from the communication controller 102) using a power module 136 including a power coil 134. The power coils 134 can be integrated within openings formed into the support system (socket) 112 that can be removably secured to a limb of the subject, directly over the sensors 118A, 118C. The support system (socket) 112 can be formed from light weight material that is resistant to radially inward compression, such as thermoplastic-fiber composite materials. Some materials forming the support system (socket) 112 can include a polymer matrix of polypropylene, polyethylene terephthalate (PET), acrylic, and/or polymethylmethacrylate (PMMA). In some implementations, the thermoplastic material of the struts can include a fiber embedded within a polymer matrix, and the fiber may be formed from carbon, glass, or any other suitable material.

For implant systems 114 distant from the communication controller 102, wireless power modules 136 with integrated external power coils and batteries 130 can be used. The power modules 136 minimize cable usage and can be placed over the location of the electronics suite 117. The power modules 136 can be positioned directly over the electronics suite 117 and held in place by support system (an arm band) 112 that can be removably secured to a portion of a limb of the subject. The powered implants can be configured to detect, by using integrated electrodes, EMG signals transmitted by respective nerves to which they are connected, condition and time stamp the detected EMG signals, position data, add internal data, and transmit the data over the short-range network 106 (Wi-Fi) to the communication controller 102. The communication controller 102 can process the data with ML models 122 that are tuned in the training mode. The ML models 122 translate the EMG signals into prosthesis commands that are stored by the communication controller 102 for use during operational mode. The prosthetic device 110 can be powered either by the communication controller 102 over a power line 138 (or wireless) or by a separate battery. The prosthesis commands can be transmitted by to the communication controller 102 to the prosthetic device 110 over the power line 138 over the short-range network 106 (e.g., using 2.4 GHz RF link).

During the operational mode, one or more parameters (e.g., battery level, connection quality) of the communication controller 102, of the implant system 114 and of the power supply system 116 can be displayed by the GUI 120 of the user device 104 that is connected with the communication controller 102 over the short-range network 106 (e.g., using 2.4 GHz RF link). The near-field RF link can operate from more than a meter distance between the implant and communication controller. In some implementations, in settings of high electromagnetic interference, a wired antenna can be routed from the communication controller to the location of the implant for a stable and robust RF link that can minimize the impact of electromagnetic interference on the signal quality. For example, in environments with high electromagnetic interference or when the implant is shielded by a carbon fiber socket, a wired antenna can be connected from the communication controller to the implant for a more reliable RF link.

The prosthetic device 110 can be an active prosthetic device, configured as a wearable robotic device controlled by the communication controller 102. The active prosthetic devices 110 described herein incorporate parallel mechanisms to improve the performance of the motions. The parallel mechanisms couple springs and motors in a parallel kinematically redundant arrangement to configure the prosthetic devices to optimize replication of human muscular behavior. For example, the motors 140A-140J are linked to linking members 142I-142J to form a kinematic chain made up of bodies connected by various joint types. The joint types include revolute joints, prismatic joints, screw-type joints, or other joint types. The joint type may further include one or more higher pair joint types, which are represented by a combination of revolute joints, prismatic joints, screw-type joints, or other joint types. The linking members 142I-142J include actuating, compliant, passive, and/or damping elements. Actuating linking members include one or more of the joints and are moved by an active component, such as a respective motor actuated by a control signal received from the communication controller 102. Compliant linking members include one or more of the joints configured as a compliant element, such as a spring and can generally be moved in association with a movement of an actuating linking member. Passive linking members can include passive joints that are independent of a controlling element, missing an associated motor. Damping linking members can include one or more of the joints configured to be controlled by a damping component, such as a dashpot.

During training mode, the user device 104 can be connected to the communication controller 102 with another communication link (e.g., RF link or Wi-Fi link) separate from the communication link used by the communication controller 102 to communicate with the sensors 118A, 118B, 118C. The user device 104 can include prestored prosthetic control scenarios or can make a connection to the server system 108, using a cell phone link, to provide access to training mode anywhere the cell phone service is available, to access prosthetic control scenarios. To train the communication controller 102, the GUI 120 of the user device 104 displays prosthetic control scenarios 124 (e.g., movement videos) that the user attempts to execute to generate corresponding EMG signals. The corresponding EMG signals are processed by the communication controller 102 to train the ML models 122 for generating prosthetic device commands for each of the displayed prosthetic control scenarios 124. In some implementations, the corresponding EMG signals are sent to the server system 108 that is configured to use the EMG signals to train the ML models 122 for generating prosthetic device commands for each of the displayed prosthetic control scenarios 124.

In response to determining, by the communication controller 102, that training is complete, the machine learning parameters including the prosthetic device commands are stored by the communication controller 102 for use during operational mode as control signals. In the example context in which the EMG data is processed by the server system 108, in response to determining that training is complete, the user device 104 can be configured to interrupt the connection to the server system 108, which is not used during operational mode. During the operational mode, the communication controller 102 can transmit the control signals patching particular EMG signals to the prosthetic device 110. The prosthetic device 110 can actuate, in response to the received control signals, one or more motors 140A-140J of the prosthetic device 110 to perform one or more movements (e.g., a series of coordinated movements) corresponding to the prosthetic control scenarios 124.

The system incorporated additional satellite IMUs 121, which can be located on the communication controller 102, surfaces of the prosthetic device 110, or exoskeleton components. These IMUs 121 provide supplementary positional and motion data to enhance the control system's accuracy and efficiency. One or more IMUs 121 integrated into the communication controller 102, mounted on the arm or leg can serve as an alternative to implant based IMUs 121 to reduce power consumption and minimize RF bandwidth requirements as the IMU 121 of the communication controller 102 provides similar positional data to that of the implants. An IMU 121 mounted on the back of the prosthetic hand provide signals that can be processed by the communication controller 102 to prevent over-rotation, under-rotation, or improper alignment of the wrist. The IMU 121 mounted on the back of the prosthetic hand can be particularly beneficial for systems lacking direct positional feedback mechanisms. An IMU positioned on the exoskeleton provides critical feedback to the communication controller, facilitating the system to maintain a targeted posture or position, such as keeping the user upright during movement or stabilization tasks. The additional IMUs 1216 enhance the system's ability to provide precise responsive control, improving both functionality and user safety.

### System for Controlling Multiple Prosthetic Devices Using Implantable Systems

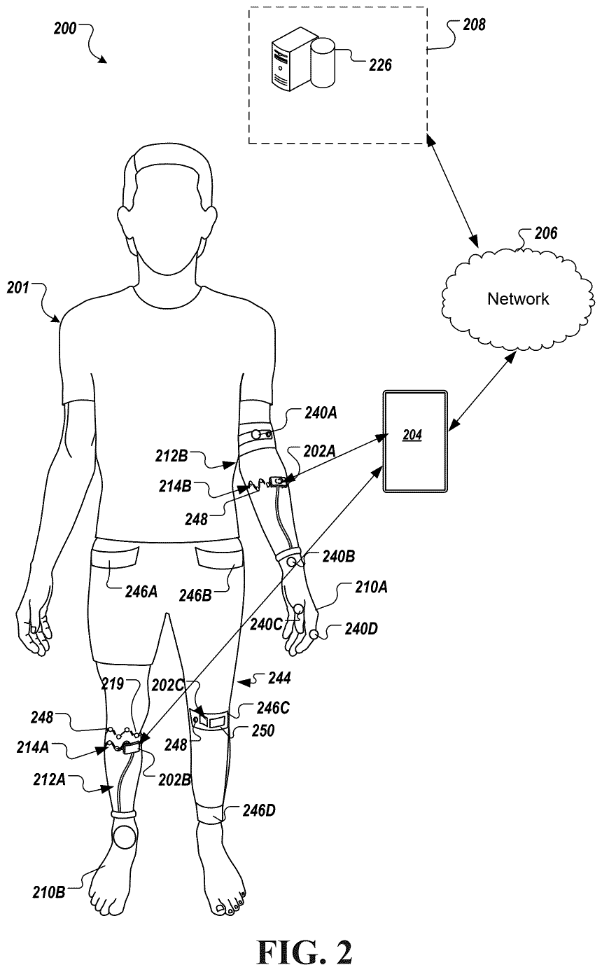

FIG. 2 depicts a block diagram of an example system 200 for controlling prosthetic devices 210A, 210B, in accordance with some example implementations. The illustrated example system 200 can include any of the components of the example system 100, described in detail with reference to FIG. 1 (e.g., a communication controller 102, a user device 104, a network 106, a server system 108, a prosthetic device 110, a support system 112, an implant system 114, and a power supply system 116) arranged in a different configuration. In particular, example system 200 includes a configuration that describes the use of multiple communication controllers 202A, 202B, 202C (e.g., similar to the communication controller 102, described in detail with reference to FIG. 1), a user device 204 (e.g., similar to the user device 104, described in detail with reference to FIG. 1), a network 206 (e.g., similar to the network 106, described in detail with reference to FIG. 1), a server system 208 (e.g., similar to the server system 108, described in detail with reference to FIG. 1), multiple prosthetic devices 210A, 210B (e.g., similar to the prosthetic device 110, described in detail with reference to FIG. 1), support systems 212A, 212B (e.g., similar to the support systems 112, described in detail with reference to FIG. 1), implant systems 214A, 214B (e.g., similar to the implant systems 114, described in detail with reference to FIG. 1), one or more power supply systems 216A, 216B and, optionally, an exoskeleton 244. In exoskeleton applications, the communication controller 202A, 202B, 202C processes EMG signals to actuate respective motorized joints. Wireless IMUs attached to the exoskeleton 244 can provide additional positional data for more accurate control.

The prosthetic devices 210A, 210B can be configured to replace a missing limb of a subject 201 and the exoskeleton 244 can be configured to assist a movement of multiple actuatable joints 240A (e.g., elbow), 240B (e.g., wrist), 240C (e.g., finger), 240D (e.g., finger) of an existing limb of the subject 201 using a respective communication controller 202C. In some implementations, the prosthetic devices 210A, 210B include a robotic foot orthotic, a robotic leg orthotic, a robotic ankle orthotic, a robotic knee brace, a robotic arm brace, a robotic leg brace.

In some implementations, the exoskeleton 244 is, but is not limited to, a hip exoskeleton, a knee exoskeleton, an ankle exoskeleton, and/or a multiple joint exoskeleton. In some implementations, the exoskeleton 244 is, but is not limited to, a soft wearable robot composed of a textile. In some implementations, the exoskeleton 244 includes an external rigid structure 246A, 246B, 246C, 246D that can be attached to at least a portion of an elastic structure 218 configured to comfortably cover all or a part of the subject's body. The rigid structure 246A, 246B, 246C, 246D can include one or more sensors 218 and the muscle actuation interface 250. The sensor(s) 218 can detect electrical signals and/or other information generated by the nerves when the subject 201 moves or attempts to move a body area of interest. For example, the sensor(s) 218 may detect a neuronal action potential (hereinafter referred to as a “nerve signal”) generated by the subject 201. Alternatively, or additionally, one or more of the sensors 218 may detect the user's pulse rate, blood pressure, temperature, combinations thereof, muscle response, and the like. While not limiting, all or some of the sensors 218 can be configured to detect neural signals generated by the subject 201 that are simultaneously measured with EMG data and IMU signals to improve accuracy and adaptability of control decoding. The sensors 218 operate to detect a neural signal generated by the subject 201 when the subject 201 moves or attempts to move a part of his or her body by operating one or more skeletal muscles and/or muscle groups. The sensor(s) 218 can transmit the detected signals to the communication controller 202C that generates control signals for the muscle actuation interface 250. The muscle actuation interface 250 generally functions to receive actuation signals from the controller 202C and apply these actuation signals to one or more muscles/muscle groups within the body area of interest. In particular, the muscle actuation interface 250 transmits the actuation signal from the controller 202C to one or more muscles/muscle groups participating in the movement of the body region of interest, for example, through the actuation of one or more muscles. The muscle operation interface 250 can transmit electrical signals to one or more motor nerves of a muscle/muscle group participating in movement and/or stabilization of a body region of interest.

As shown in FIG. 2, the example system 200 includes multiple prosthetic devices 210A, 210B and an exoskeleton 244 attached to a subject 201. Each of the plurality of prosthetic devices 210A, 210B can be controlled by a respective communication controller 202A, 202B based on wireless EMG signals received from a respective set of one or more implants of the implant systems 214A, 214B including an array of sensors housed within a sensor strand 219 having a tubular body. The communication controller 202A, 202B can be configured to process the EMG signals received from the respective implants and positional data from IMUs integrated in a respective communication controller 202A, 202B, respective implants of the implant systems 214A, 214B or a respective on the prosthetic device 210A, 210B.

The communication controller 202A, 202B can be configured to generate independent prosthetic control signals based on the received EMG signals and positional data and send the prosthetic control signal to a respective prosthetic device 210A, 210B, in an operational mode. In some implementations, the communication controllers 202A, 202B, 202C of the example system 200 facilitate positional awareness of the prosthetic devices 210A, 210B and/or the exoskeleton 244. A BLE-connected Inertial Measurement Unit (IMU) can be attached to the prosthetic devices 210A, 210B and/or the exoskeleton 244 to determine the respective positions and movements, either in absolute terms or relative to the respective communication controllers 202A, 202B, 202C. The IMU can be strategically placed on various parts of the prosthetic devices 210A, 210B, such as the wrist, back of the hand, or even a specific finger (e.g., the ring finger), to provide accurate motion and location data. The additional feedback enhances the overall control system's accuracy and responsiveness, enabling more natural and intuitive prosthetic movement.

The short-range communication network can be configured to prevent signal interferences (including, but not limited to crosstalk) between implant systems 214A, 214B and non-associated communication controllers 202B, 202A as well as signal interferences between communication controllers 202A, 202B and non-associated prosthetic devices 210B, 210A. The prevention of signal interference can include transmission channel selection, signal transmission gating based on signal frequency and/or time modulation.

Although communication controllers 202A, 202B, 202C can provide independent (non-interfering) control of respective prosthetic devices 210A, 210B or the exoskeleton 244, in some implementations, actuation of two or more prosthetic devices 210A, 210B and/or the exoskeleton 244 can be provided by a single communication controller 202A or can be coordinated by a single communication controller 202A designated as a master communication controller 202A, in a coordinated operation mode. In some implementations, the coordinated operation mode can be enabled by the subject 201 in a training mode, by selecting training using prosthetic control scenarios corresponding to the coordinated operation mode, involving actuation of a combination of prosthetic device(s) 210A, 210B and/or the exoskeleton 244. In the coordinated operation mode, the master communication controller is configured to process the EMG signals and to actuate at least a portion of the exoskeleton 244 (e.g., motorized braces) to execute an augmented movement synchronized with an actuation of one or more prosthetic device 210A, 210B. In some implementations, the power supply system 216 provides power for all communication controllers 202A, 202B, 202C or the power supply system 216 can include multiple power supply systems 216A, 216B, 216C (including multiple external battery packs) separately providing power the communication controllers 202A, 202B, 202C, to minimize wired connections between different regions of the subject 201. The communication controllers 202A, 202B, 202C can be configured to supply transcutaneous radio frequency power to one or more of their respective implant system 214A, 214B through a power coil and can include power lines to supply power to the prosthetic device 210A, 210B, as described in FIG. 1.

### System Configuration Including Multiple Patient Implantable Systems

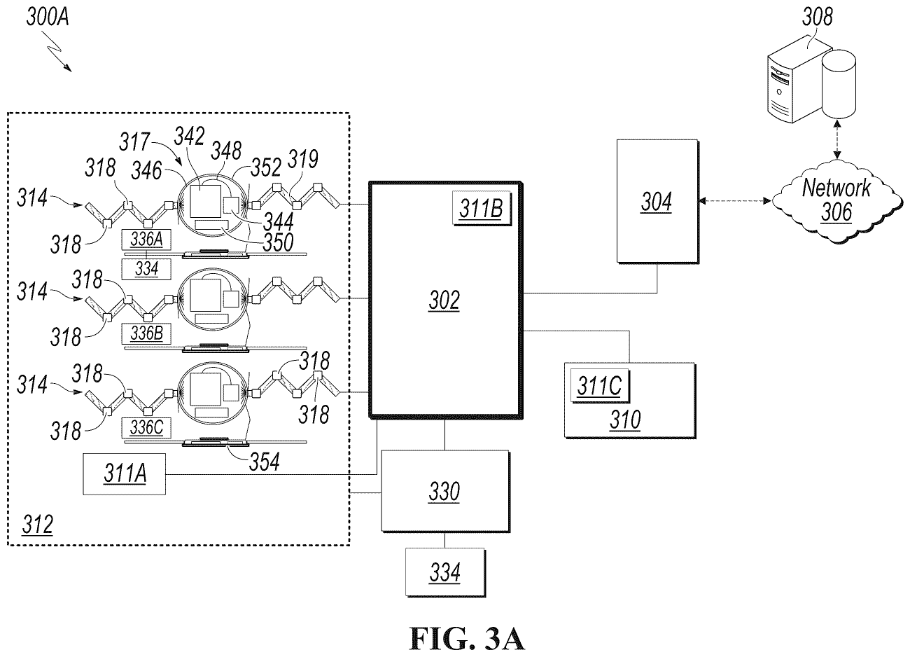

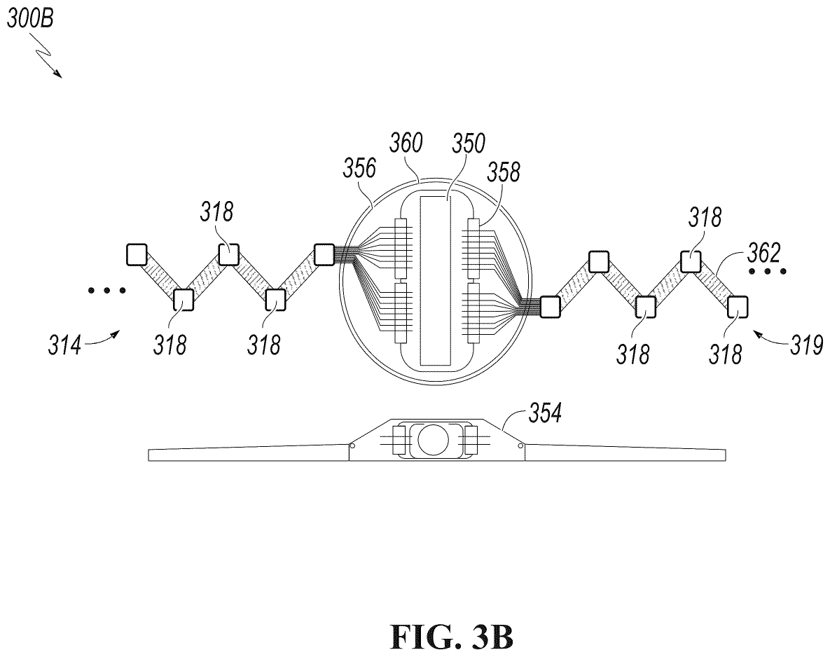

FIG. 3A depicts a schematic diagram illustrating an example system 300A including multiple patient implantable systems 314, in accordance with some example implementations. The example system 300A can include any of the components of the example system 100, described in detail with reference to FIG. 1 (e.g., a communication controller 102, a user device 104, a network 106, a prosthetic device 110, a support system 112, an implant system 114, a power supply system 116, and, optionally, a server system 108) arranged in a different configuration. In particular, example system 300A includes a configuration that facilitates use of sensor data by a communication controller 302 (e.g., similar to the communication controller 102, described in detail with reference to FIG. 1) that can be coupled to a user device 304 (e.g., similar to the user device 104, described in detail with reference to FIG. 1), a network 306 (e.g., similar to the network 106, described in detail with reference to FIG. 1), a server system 308 (e.g., similar to the server system 108, described in detail with reference to FIG. 1), a prosthetic device 310 (e.g., similar to the prosthetic device 110, described in detail with reference to FIG. 1), IMUs 311A, 311B, 311C (e.g., similar to the IMUs 121, described in detail with reference to FIG. 1). The communication controller 302 can receive the sensor data from implantable systems 314 (e.g., similar to the implants 114, described in detail with reference to FIG. 1). The implantable systems 314 can include an electronics suite 317 and sensors 318 (e.g., similar to the sensors 118A-118C, described in detail with reference to FIG. 1) attached to or included in a straight or zig zag sensor strand 319.

The electronics suite 317 can be designed to monitor and transmit patient data. The electronics suite 317 can include an application specific integrated circuit 342, a Bluetooth low energy module 344, an NFC circuit 346, an antenna 348, an optional battery 350, and a case 360. The application specific integrated circuit 342 can include a custom-designed chip that handles specific tasks such as signal processing, data acquisition, and power management to facilitate efficient operation and integration of the various components within the implantable system 314. The Bluetooth® low energy module 344 can include facilitate wireless communication with external devices, such as the user device 302 or medical monitoring equipment. The Bluetooth low energy module 344 has low power consumption, extending the life span of the implantable system 314. The NFC circuit 346 can be configured for short-range wireless communication, typically used for data transfer and device configuration. The NFC circuit 346 facilitates secure communication when the implantable device is in close proximity to an NFC reader. The antenna 348 antenna facilitates wireless communication at the 2.4 GHz frequency, commonly used for Bluetooth and Wi-Fi for reliable data transmission between the implantable system 314 and external receivers, such as the user device 302. The optional battery 350 can provide power to the implantable system 314. The electronics suite 317 can be encapsulated in a polyimide circuit 352 with an approximately 15 mm diameter and 3 mm height, which offers flexibility and biocompatibility, ensuring safe and long-term operation within the body. The electronics suite 317 can be connected to a reference electrode 354 that is used to measure background (electrical) signals within the body to provide a stable reference point for accurate data acquisition and monitoring of patient data. The electronics suite 317 can be connected to a power source 330 (e.g., similar to the battery 130, described in detail with reference to FIG. 1), and a power coil 334 (e.g., similar to the power coil 134, described in detail with reference to FIG. 1). A satellite IMU 360 can connect to the communication controller 302 with a near-field communication (NFC) or a mid-field communication (proprietary RF, such as Bluetooth® Low Energy link). The case 360 can provide non-hermetic closure if the electronics suite 317 does not contain a battery. The non-hermetic patient implantable system can include features that ensure the effectiveness and reliability of the non-hermetic closure. Choosing the right polymers is crucial for the success of non-hermetic systems. Polymers such as polyurethane, silicone, and polyethylene can provide protection against moisture and corrosion, while being biocompatible and flexible. For example, the case 360 designed for non-hermetic closure can have the sensors 318 encapsulated in: parylene, silicone elastomers, polyimide (Kapton), or liquid crystal polymers. The case 360 can be designed to adequately protect the electronics suite 317 by coating (e.g., dip-coating, spray-coating, or overmolding process) or encapsulating the circuits to shield the electronic components 342, 344 from bodily fluids and potential contaminants. The non-hermetic patient implantable system can be tested to validate the performance and durability of the case 360. The testing includes conducting accelerated aging tests, biocompatibility assessments, and environmental exposure evaluations. The case 360 can be implemented in conjunction with comprehensive maintenance protocols to address frequent technical revisions, such as scheduled check-ups and replacement plans that facilitate the ongoing functionality of the example system 300A. The case 360 can be flexible, allowing for conformance to anatomical structures during or after implantation.

The example system 300A can be assembled (setup) after the sensors 318 are implanted (e.g., attached to a nervous system of a subject) and the inflammation subsided. In the case where a prosthesis socket is required and the sensors 318 are attached to a support system 312 (e.g., a sensor strand 319), the support system 312 can be included in the example system 300A. The support system 312 can include one or more IMUs 311A. In some implementations, one or more sensors 318 and IMUs 311A, 311B, 311C can be away from the socket, being attached to an arm band or another retaining device to hold a respective power module 336A, 336B, 336C proximal to (approximately above) the respective patient implantable system 314. In some implementations, one or more IMUs 311B, 311C can be away from the support system 312, being attached to the communication controller 302 and/or the prosthetic device 310. The controller's IMU 311B and additional wireless IMUs 311C (e.g., on the back of the hand) factor into the control solution to replicate natural human movement, such as wrist positioning.

The communication controller 302 and/or the user device 304 can be used to scan each component of the example system 300A including the sensors 318 and IMUs 311A, 311B, 311C using a near field communication (NFC) of the network 306. Each component can have an NFC identifier tag for the purposes of cybersecurity and communications. For components, such as a third-party prosthetic device 110 that does not have a tag, a passive tag can be provided that identifies the device in use, in that way only approved devices are integrated within the example system 300A. Once all the unique identifiers are identified by the communication controller 302 and/or the user device 304, the communication controller 302 can store the unique identifiers. If the power coils 334 have not been inserted into the socket, the GUI of the user device 304 can display a visual guide to align and place the coils in the proper location. In addition, the GUI of the user device 304 can also support alignment of the power modules 336A, 336B, 336C held in place by arm bands.

The user device 330 can display sensor data (e.g., EMG signal and position data) and healthcare protocols (e.g., prosthetic control scenarios) that can be stored by the user device 330. The selected prosthetic control scenarios can be accompanied by instructional videos to synchronize EMG signal and position data acquisition. A machine learning training process can include locally processing, by the communication controller 302, or remotely processing, by the remote server system 308, the EMG data and the position data to train the machine learning model. The communication controller 302 can perform a training data validation. Inline validation ensures system accuracy by subsampling rest periods (when the user is not moving) to exclude poor-quality data.

For the remote processing, the example system 300A can be assembled (setup) to configure the components to securely communicate with each other and ensure compatibility of the system. During remote processing, the setup of the example system 300A can be performed using a server system 308 (e.g., configured to act as a phantom key server and configurator). The server system 308 can provide a cloud service to configure the example system 300A and provide secure keys and connection (RF channels and/or Wi-Fi addresses) for the components of the example system 300A. After the unique identifiers to the server system 308 are stored by the server system 308, any firmware updates can also be performed and the channels and/or addresses can be updated.

In response to determining that the setup of the example system 300A was successfully completed, a training mode can be initiated by the user device 304. The user device 304 can be connected to the communication controller 302 with a fast Wi-Fi link of the network 306. The user device 304 can make a connection to the server system 308 with a cell phone link, enabling the user to train anywhere where cell phone service is available. To train the communication controller 302, the user device 304 can present demonstration videos that the subject follows along with. The corresponding tagged EMG data is sent up the server system 308 (cloud) where the ML algorithms are trained. Once training is complete, the ML parameters are stored by the communication controller 302 for use during operational mode (also referred to as run mode). The communication controller 302 does not need connection to the server system 308 during normal control mode, as described with reference to FIG. 3B.

FIG. 3B shows a schematic diagram of an example implantable battery-powered implantable sensor strand 300B, in accordance with some example implementations.

The example implantable battery-powered implantable sensor strand 300B can include an electronics suite 317, sensors 318, and a sensory strand 319. The electronics suite 317 can be designed to monitor and transmit patient data collected by the sensors 318. The electronics suite 317 can designed for robust and reliable performance in biological (subcutaneous) medium. The electronics suite 317 can include any of the components described with reference to FIG. 3A, a battery 350, a battery charging coil 356, multi (8)-pin feedthroughs 358, and a case 360.

The battery 350 can be a Lithium-ion pin-type battery. The battery 350 can have a capacity around 13 mAh when charged to 4.2 V1. The battery 350 can have a high-strength metal exterior case for reliability, stainless steel tab with tin (Sn) plating. The battery 350 can provide power to the electronics suite 317, facilitating long-lasting and stable energy supply. In some implementations, the electronics suite 317 can be designed to have a consumption that can be sufficiently supported throughout a day by a miniaturized battery 350 that provides power for at least 8 hours at a 2 mA dissipation for 32 channels (currently at 4 mA for 16 channels) achievable with the following configuration: approximately 4 Mbit 2.4 GHz proprietary radio link with a 1:1 linkage with the communication controller (e.g., communication controller 102 described with reference to FIG. 1) within 1 m; approximately 15-450 Hz bandwidth sampled at 1250 Hz but the bandwidth and sample rate can be increased in software for exploratory research work; and a data compression ratio boosted 4:1 to reduce power.

In some implementations, the example implantable battery-powered implantable sensor strand 300B is powered over a wireless power system using a magnetic link. The battery charging coil 356 can provide charging power around a domed silastic puck. The battery charging coil 356 can include a wire that can be shared with two electrodes, or 2 of the 32 channels can be utilized for power input. In some implementations, there is no significant energy storage on the implantable device; thus, the wireless link is configured to be on constantly while the system is in use. In some implementations, the output voltage of the power receiver coil is rectified and smoothed, resulting in an unregulated voltage from which all other power supplies are generated. In some implementations, the electronics module further includes an integrated current, voltage, and power measurement circuit configured to measure the voltage received by the implantable device and the current drawn by it. In some implementations, measuring the voltage received and the current drawn enables an alignment assistance function of the wearable device and closed-loop power control, if necessary.

In some implementations, the electronic suite 317 can include a non-rechargeable battery 350 that can be replaced at a set time (e.g., every 5-10 years) or a rechargeable battery that can be recharged using an arm band with a charger for overnight charge. The non-rechargeable battery can function as a backup for the rechargeable battery.

The multi (8)-pin feedthroughs 358 can be made from high-quality materials to ensure hermetic sealing and reliable electrical connections. The multi (8)-pin feedthroughs 358 can facilitate the passage of electrical signals from the sensory strand 319 through the case 360 while maintaining the integrity and hermeticity of the enclosure. The case 360 can be a Titanium (Ti) case. The case 360 can provide the electronics suite 317 with strength, corrosion resistance, and biocompatibility. The case 360 can provide a durable and protective enclosure for the electronics suite 317, such that the components of the electronics suite 317 are shielded from external environmental factors. The case 360 can be used to house the battery 350, feedthroughs 358, and other electronic components, offering protection and structural integrity. The case 360 can include a silicone over mold to 20 D×4 mm where the size is driven by the feedthrough, custom feed throughs can reduce size to ˜16 D×4 mm. If the electronics suite 317 includes a battery 350, the electronics suite 317 is hermetically sealed by the case 360 to prevent chemical leakage and ensure patient safety. The case 360 can also provide a durable and protective enclosure for the reference electrode 354. The reference electrode 354 can be integrated with a flexible printed circuit board (flex PCB) designed to provide stable and reliable measurements in the biological medium. The reference electrode 354 can be approximately 25 mm by 4.5 mm.

The example implantable battery-powered implantable sensor strand 300B can include a straight or zig zag sensor strand 319 that can be made of lead wires or traces 362 on a polyimide substrate 364. The lead wires or traces 362 in a straight configuration, can be aligned in parallel between the sensors 318 and the feedthroughs 358. In a zigzag configuration, the lead wires or traces 362 follow a back-and-forth pattern. The zigzag configuration can include the sensors 318 at each inflection point and, optionally, at one or more points (example middle point) forming two or more rows of sensors. The lead wires or traces 362 can be embedded on the polyimide substrate 364. The polyimide substrate 364 can be flexible, durable, and biocompatible, providing resistance to bodily fluids and mechanical stress. The polyimide substrate 364 can reduce electromagnetic interference by balancing inductance and capacitance.

### Shape Design for Example Patient Implantable System during Operations

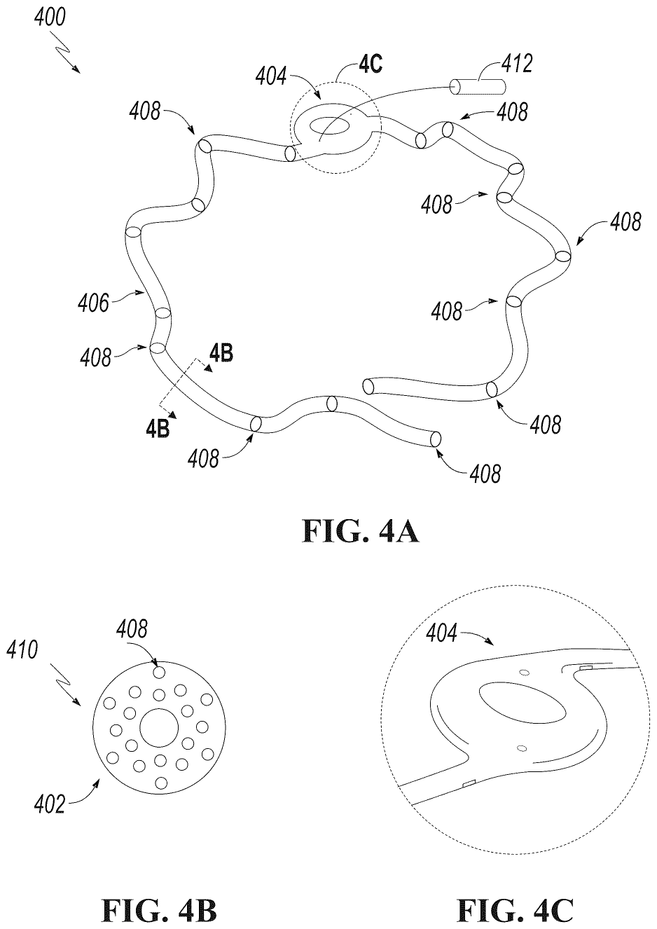

FIG. 4A depicts a schematic diagram illustrating an example patient implantable system 400, in accordance with some example implementations. FIG. 4B depicts a schematic diagram illustrating a cross section 402 of the example patient implantable system 400, in accordance with some example implementations. FIG. 4C depicts a schematic diagram illustrating an example electronics suite 404 of the example patient implantable system 400, in accordance with some example implementations.

The example patient implantable system 400, as shown in FIG. 4A forms a squiggly bracelet having the example electronics suite 404 in a middle portion between two implantable sensor strands 406. As shown in FIGS. 4A and 4B, the example electronics suite 404 can have a disc shape with smoothly rounded edges. The example patient implantable system 400 includes the example electronics suite 404 and an implantable sensor strand 406. The implantable sensor strand 406 includes a tubular design (as shown in FIG. 4B) or a flattened design. The overmolded cross section, as shown in FIG. 4B, may be round with a circumferential electrode to receive EMG signals from all directions, or flat or oval where the electrode can be faced downward for direct access to EMG, or upward to record a reference signal. The implantable sensor strand 406 can have a flexibility that prevents unassisted insertion in the epimysial between the facia and the muscle alone. Stiffness can increase with a number of electrodes. The implantable sensor strand 406 can have a set number of electrodes, such as 4, 8, 16, or 32 electrodes. The implantable sensor strand 406 includes multiple sensors 408 along the length of the sensor strand, along with implantable electronics. Each sensor 408 is spaced out from one another within the interior of the tube. Each sensor 408 is electrically coupled to an electrode on the exterior of the tube.

The sensor strand 406 can be built from a multi-filer lumen 410 with separate lumens for each electrode wire and a large central lumen 410 for insertion of a stylet that is used for implantation and shape forming. The lumen 410 is over-molded with silicon to establish a shape that loosely grips around the arm like a loose bracelet. The inner lumen 410 can fill with fluid that does not affect sensor data transmission because the wires connecting the sensors to the electronics suite are coated. The lumen 410 can have a diameter of substantially 1.1 to 1.6 mm and an outer diameter of substantially 1.3 to 1.8 mm.

In some implementations, the patient implantable system 400 can be inserted using an insertion stylet 412. The insertion stylet 412 can guide the patient implantable system during implantation into a set position. The insertion stylet 412 can be designed to provide more shape or rigidity to the implanted device. The insertion stylet 412 can remain in the patient implantable system permanently to facilitate removal of the patient implantable system. The insertion stylet 412 can have several sizes or curvatures of positioning stylets to adapt to the patient anatomy. The stylet 412 can be inserted into the array to stiffen it so that it can be directly inserted between tissue layers. The stylet 412 can include 14 gage needle for insertion and steering. Once positioned the stylet 412 is removed leaving the array in place. The insertion procedure can be separately performed for each portion of the sensor array (e.g., on the left and right side of the electronics suite 404).

The removable stylet 412 can be positioned within a flexible conduit configured for implantation or subcutaneous positioning between tissue planes of a limb. The stylet 412 is sufficiently stiff to facilitate insertion of the conduit between anatomical tissue planes. In some implementations, the removable stylet 412 is removable after placement to allow the conduit to assume a neutral or more conformable shape governed by the flexibility of the conduit material. In some implementations, the removable stylet 412 is replaceable with a second, more flexible stylet 412 designed to impart a gentle curvature that hugs the contours of a limb, such as an arm or leg, for improved anatomical conformity. A selection of stylets 412 with different pre-formed curvatures and stiffness levels adapted to conform to various anatomical structures and appendage sizes designed to fit anatomical features of a patient receiving the implant (e.g., diameter of the patient limb). The stylet 412 is composed of a shape-memory material, such as nitinol. The stylet 412 is insertable in a substantially straight configuration. The stylet 412 in response to exposure to body temperature or physiological conditions, assumes a curved or coiled geometry to conform to the surrounding anatomical structure.

### Shape Design for Example Patient Implantable System with a Disc Shaped Electronics Suite during Insertion and Operation

FIG. 5A depicts a schematic diagram illustrating another example patient implantable system 500, in accordance with some example implementations. The example patient implantable system 500 includes an electronics suite 502 and a sensor strand 504. FIG. 5B depicts a schematic diagram illustrating a longitudinal section of the example sensor strand 504 in a straight configuration, in accordance with some example implementations. FIG. 5C depicts a schematic diagram illustrating the example patient implantable system 500 of FIG. 5B during insertion, in accordance with some example implementations. FIG. 5D depicts a schematic diagram illustrating a longitudinal section of the example sensor strand 504 in a bent configuration, in accordance with some example implementations. FIG. 5E depicts a schematic diagram illustrating the example patient implantable system 500 during operation, in accordance with some example implementations. The electronics suite example can be housed in a disc-shaped housing; or the housing can be of a different shape with rounded edges. A distal end of the implantable sensor strand 504 can be coupled to the electronics suite 502. The sensor strand 504, as shown in FIGS. 5A-5E, includes a flattened, flexible, multi-sensor strand (e.g., including an array of sensors 508).

FIGS. 5A-5D show an implantable sensor strand that can be implanted with a minimally invasive electrode insertion procedure. The implantable sensor strand 504 is implantable subcutaneously. For example, the implantable sensor strand 504 can be implanted on top of muscle surface (being coupled to the epimysium of the subject) to detect EMG signals transmitted by the nerves to muscle fibers. The sensor strand 504 can include a tube 510 that can bend and flex during the implanting procedure. The sensors 508 within the tube 510 can be positioned in two dimensions around the implantation site.

The sensor strand 504 includes a straight-to-staggered mechanism being configured to have a first shape during insertion (as shown in FIGS. 5B and 5C) and a second shape during operation (as shown in FIGS. 5D and 5E), the second shape arranging the plurality of sensors in two or more rows. The straight-to-staggered (shape actuation) mechanism can include a tightening rope (draw string) 506 that cinches the sensor strand 504 across its length. The tightening rope (draw string) 506 includes a draw string (made of plastics-type material) used to pull the implantable sensor strand into a staggered circular layout forming an electrode array bracelet. The squiggly configuration can form multiple loops with substantially equal amplitudes and angles that stagger the sensors 508 into two or more rows. For example, the bending and flexing of the sensor strand 504 generates a multi-dimensional array of sensors 508. The sensor strand 504 applies zero force on the electronics suite 502. The sensor strand 504 can include internal scaffolding to support structures (e.g., elastic bands or strips) that ensure that the sensor array squiggles by forming inflection points at preset locations and across a single plane.

For example, after being inserted, the implantable sensor strand 504 can stagger or flex or bend in multiple directions, such that the sensors 508 become displaced relative to each other, thereby creating a two-dimensional or a three-dimensional array of sensors 508. In some implementations, a pre-formed bend in the implantable sensor strand 504 can be straightened, for the purpose of implantation, with the use of a straightening element. For example, the sensor strand 504 can be printed as a pigtail structure out of a flexible material to incorporate more complex internal support features as part of one solid structure. Upon removal of the straightening element, the implantable array can re-acquire a respective bent conformation. The pre-formed bend in the implantable sensor provides a low material, high surface area sensor array that can be implanted with a minimally invasive process. The example patient implantable system 500 provides a minimized material inserted in the body that simultaneously maximizes surface area covered with that material.

### Shape Design for Example Patient Implantable System with a Tubular Shaped Electronics Suite

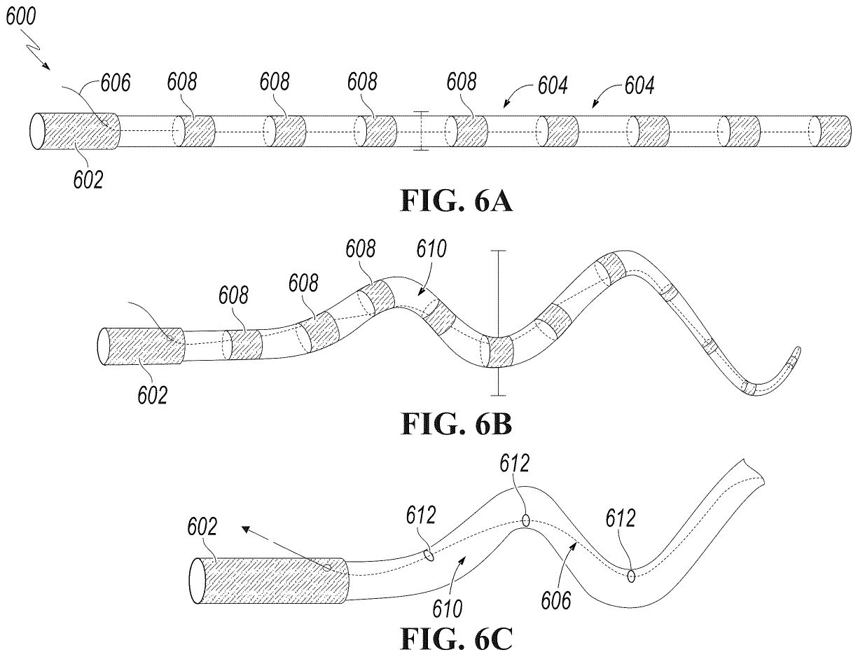

FIG. 6A depicts a schematic diagram illustrating another example patient implantable system 600, in accordance with some example implementations. The example patient implantable system 600 includes an electronics suite 602 and a sensor strand 604. FIG. 6B depicts a schematic diagram illustrating a longitudinal section of the example sensor strand 604 of the example patient implantable system 600 of FIG. 6A, in accordance with some example implementations. FIG. 6C depicts a schematic diagram illustrating another longitudinal section of the example sensor strand 604 of the example patient implantable system 600 of FIG. 6A, in accordance with some example implementations.

The electronics suite 602 can house electronics in a cylindrical housing with rounded edges having a diameter substantially equal to or greater than the diameter of the example sensor strand 604. A distal end of the implantable sensor strand 604 can be coupled to the electronics suite 602. The sensor strand 604, as shown in FIGS. 6A-5C, includes a tubular or flattened, flexible strand (e.g., including an array of sensors 608) 610.

The sensor strand 604 can be configured to be adjustable using a straight-to-staggered mechanism via tightening rope (draw string) 606 that cinches the strand 610. The tightening rope (draw string) 606 can be coupled to a shape retention feature 612 that can be integrated across a portion or the entire length of the strand 610. The shape retention feature 612 facilitates a setting of the squiggle parameters (e.g., position of inflection points where the strand changes direction). The shape retention feature 612 can include rings through which the tightening rope (draw string) 606 is guided during the execution of the straight-to-staggered mechanism. The rings guide the tightening rope 606 to move smoothly and to maintain a selected shape and dimension of the strand to match a limb diameter. In some implementations, the final geometrical characteristics of the sensor strand 604 (e.g., squiggle parameters, length of the sensor strand 604 and diameter of the bracelet formed by the sensor strand 604) can be set based on how far the tightening rope (draw string) 606 is pulled out.

### Shape Design for Example Patient Implantable System with Adjustable Geometry

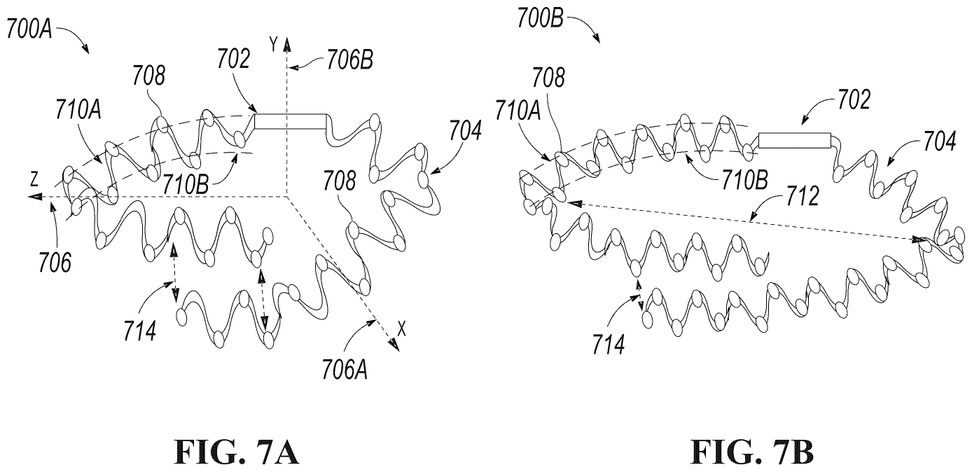

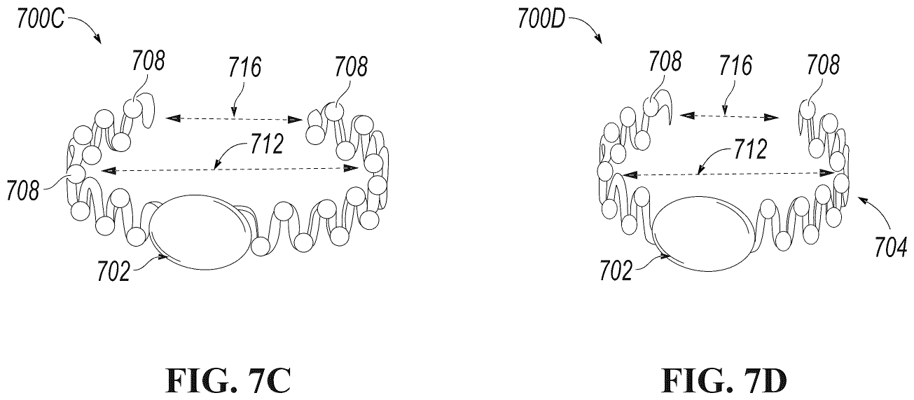

FIG. 7A depicts a schematic diagram illustrating an example patient implantable system 700A, in accordance with some example implementations. FIG. 7B depicts a schematic diagram illustrating another example patient implantable system 700B, in accordance with some example implementations. FIG. 7C depicts a schematic diagram illustrating another example patient implantable system 700C, in accordance with some example implementations. FIG. 7D depicts a schematic diagram illustrating another example patient implantable system 700D, in accordance with some example implementations.

FIGS. 7A-7D show example patient implantable systems 700A-700D including an electronics suite 702 located between two sections of a sensor strand 704. The example patient implantable systems 700A-700D form adjustable size squiggly bracelets designed to provide both flexibility and size adjustment across the three-dimensional space (XYZ 706A-706C) for optimization of patient data collection. The sensor strand 704 includes multiple sensors 708 that are distributed along two parallel circular rows 710A, 710B that surround an inner circumference of a patient limb (arm or leg).

The example patient implantable systems 700A and 700B can form coiled adjustable size squiggly bracelets of larger or smaller diameters 712 that can be adjusted to match a limb diameter, by modifying the angles of the loops of the sections of a sensor strand 704 (e.g., by shortening or extending the draw string, as described with reference to FIGS. 5 and 6) to wrap around in a spiral or helical pattern. The sections of a sensor strand 704 can be configured to have the ends circumferentially overlapping 714, such that at least a portion of the sections of a sensor strand 704, wrap around a same portion of the circumference of the limb on top of each other, as shown in FIGS. 7A and 7B. The example patient implantable systems 700C and 700D can form open-end adjustable size squiggly bracelets of larger or smaller diameters 712 that can be adjusted by modifying the angles of the loops (zigzags) of the sections of a sensor strand 704 (e.g., by shortening or extending the draw string, as described with reference to FIGS. 5 and 6). For example, for a small limb the example patient implantable systems 700C and 700D can form open-end adjustable size squiggly bracelets with a greater number of loops (zigzags) to fit within the circumference of the limb without an overlap. For a larger limb diameter, the example patient implantable systems 700C and 700D can form open-end adjustable size squiggly bracelets with a lower number of loops (zigzags) forming a straighter profile that fits around the entire limb without a gap 716 or with a minimal (e.g., less than substantially 1 cm) gap 716.

The sections of a sensor strand 704 can be configured to have the ends circumferentially adjustably distanced from each other, for example to minimize the gap 716. The circumferentially overlap 714 and the gap 716 between the ends of the sections of the sensor strand 704 can be adjusted by modifying the angles of the loops of the sections of a sensor strand 704 (e.g., by shortening or extending the draw string, as described with reference to FIGS. 5 and 6).

### Example Processes for Using a Patient Implantable System for Collecting Internal Patient Data

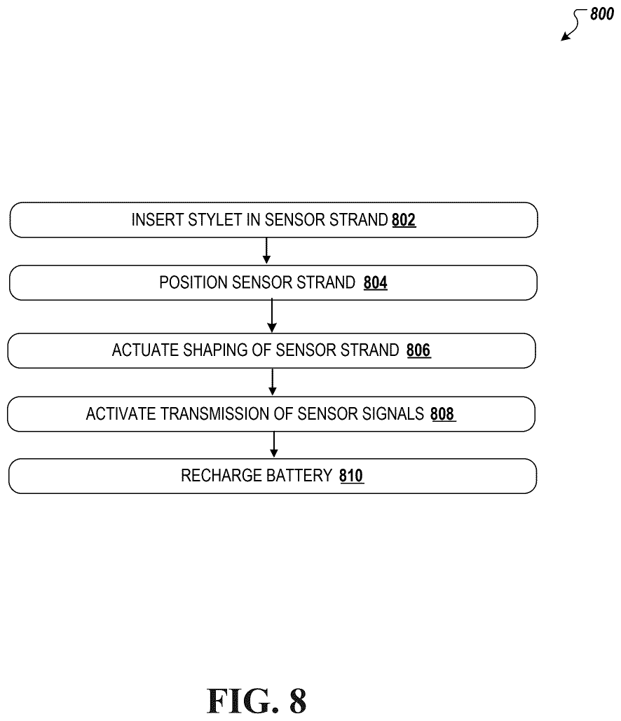

FIG. 8 depicts a schematic flow diagram illustrating an example process of inserting and using an example patient implantable system, in accordance with some example implementations.

At 802, a sensor strand of a patient implantable system is coupled with a stylet. The sensor strand is formed of a flexible material, facilitating the sensor strand to conform to the patient's anatomy and provide accurate measurements. The stylet is designed to provide shape and rigidity to the sensor strand during insertion, being made from materials like stainless steel or other biocompatible metals. The stylet can have different sizes and curvatures to adapt to various anatomical structures (e.g., limb diameter) and facilitate precise positioning with minimal invasiveness. The insertion stylet can be designed to provide shape or rigidity to the sensor strand. The stylet can be inserted between the sensor array to stiffen the sensor strand. In some implementations, the insertion of the stylet can be verified using imaging techniques like fluoroscopy or ultrasound to guide the insertion process and ensure accurate placement relative to the sensors of the sensor strand.

At 804, the patient implantable system is inserted through an incision within a subcutaneous medium, using the insertion stylet for positioning the sensor strand between tissue layers. The insertion stylet can guide the patient implantable system during implantation into a set position. The insertion stylet can be removed from the sensor strand or can remain in the patient implantable system permanently to facilitate removal of the patient implantable system. The stylet can include 14 gage needle for insertion and steering. The patient implantable systems including the electronics suite between two sections of the sensor strand can be inserted using a two-step protocol. The two-step protocol can include an insertion procedure that is separately performed for each section of the sensor strand (e.g., on the left and right side of the electronics suite). The patient implantable system is inserted having the sensor strand in a first (linear) shape to minimize tissue injury.

At 806, a shaping of the sensor strand from the first (linear) shape to the second (squiggly) shape is actuated. The straight-to-staggered (shape actuation) mechanism can include a tightening rope (draw string) that cinches the sensor strand across its length. The tightening rope (draw string) includes a draw string (made of plastics-type material) used to pull the implantable sensor strand into a staggered circular layout forming an electrode array bracelet. The squiggly configuration can form multiple loops with substantially equal amplitudes and angles that stagger the sensors into two or more rows. For example, the bending and flexing of the sensor strand generates a multi-dimensional array of sensors. The sensor strand applies zero force on the electronics suite. The sensor strand can include internal scaffolding to support structures (e.g., elastic bands or strips) that ensure that the sensor array squiggles by forming inflection points at preset locations and across a single plane.

At 808, the patient implantable system is activated. Activation of the patient implantable system includes verification of post-operative compliance after completion of recovery time to ensure absence of infection and inflammation. In some implementations, the activation of the patient implantable system includes powering the patient implantable system. Activation of the patient implantable system includes establishing communication with a communication controller, initial calibration to ensure accurate data collection by the sensors, and testing to verify that the sensor and the transmission system are functioning correctly. Activation of the patient implantable system includes configuring an external device to recognize and communicate with the implant and data transmission activation to execute continuous patient data monitoring and processing according to a set healthcare protocol. For example, the communication controller can establish and verify communication channels and in response to identifying interference significantly impacting the quality of communication, the communication controller can establish communication with the respective implant using a new channel for the transmission of a signal packet.

At 810, the battery of the patient implantable system is recharged according to a schedule. The recharging schedule can be daily (e.g., according to an overnight recharge protocol) or it can be set to be activated at a longer time interval of several years. For example, the activation of the patient implantable system can automatically activate a clock to trigger an alarm for battery recharging.

### Example Computing System For Performing Described Processes

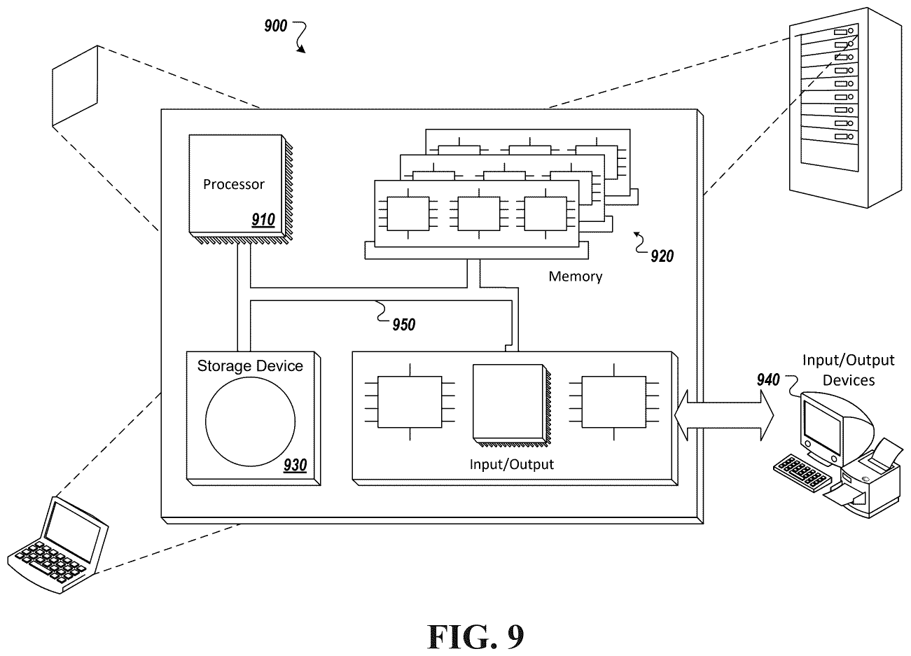

FIG. 9 depicts a block diagram illustrating a computing system 900, in accordance with some example implementations. Referring to FIG. 1, the computing system 900 can be used to implement the communication controller 102, the user device, 104, and/or any components therein.

As shown in FIG. 9, the computing system 900 can include a processor 910, a memory 920, a storage device 930, and input/output devices 940. The processor 910, the memory 920, the storage device 930, and the input/output devices 940 can be interconnected using a system bus 950. The processor 910 is capable of processing instructions for execution within the computing system 900. Such executed instructions can implement one or more components of, for example, the machine learning controller 110 and the natural language processing engine 120. In some implementations of the current subject matter, the processor 910 can be a single-threaded processor. Alternately, the processor 910 can be a multi-threaded processor. The processor 910 is capable of processing instructions stored in the memory 920 and/or on the storage device 930 to display graphical information for a user interface provided using the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, an optical disk device, or a tape device, or other suitable persistent storage means. The input/output device 940 provides input/output operations for the computing system 900. In some implementations of the current subject matter, the input/output device 940 includes a keyboard and/or pointing device. In various implementations, the input/output device 940 includes a display unit for displaying graphical user interfaces.

According to some implementations of the current subject matter, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the Internet).

In some implementations of the current subject matter, the computing system 900 can be used to execute various interactive computer software applications that can be used for organization, analysis and/or storage of data in various (e.g., tabular) format (e.g., Microsoft Excel®, and/or any other type of software). Alternatively, the computing system 900 can be used to execute any type of software applications. These applications can be used to perform various functionalities, e.g., planning functionalities (e.g., generating, managing, editing of spreadsheet documents, word processing documents, and/or any other objects), computing functionalities, or communications functionalities. The applications can include various add-in functionalities or can be standalone computing products and/or functionalities. Upon activation within the applications, the functionalities can be used to generate the user interface provided using the input/output device 940. The user interface can be generated and presented to a user by the computing system 900 (e.g., on a computer screen monitor).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable hardware processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The preceding figures and accompanying description illustrate example processes and computer implementable techniques. The environments and systems described above (or their software or other components) may contemplate using, implementing, or executing any suitable technique for performing these and other tasks. It can be understood that these processes are for illustration purposes only and that the described or similar techniques may be performed at any appropriate time, including concurrently, individually, in parallel, and/or in combination. In addition, many of the operations in these processes may take place simultaneously, concurrently, in parallel, and/or in different orders than as shown. Moreover, processes may have additional operations, fewer operations, and/or different operations, so long as the methods remain appropriate.

In other words, although the disclosure has been described in terms of certain implementations and generally associated methods, alterations and permutations of these implementations, and methods can be apparent to those skilled in the art. Accordingly, the above description of example implementations does not define or constrain the disclosure. Other changes, substitutions, and alterations are also possible without departing from the spirit and scope of the disclosure.

A number of implementations of the present disclosure have been described. Nevertheless, it can be understood that various modifications may be made without departing from the spirit and scope of the present disclosure. Accordingly, other implementations are within the scope of the following claims.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application.

Example 1. A patient implantable system for subdermal collection of patient data, the patient implantable system comprising: a sensor strand comprising a plurality of sensors housed within a tubular body, the plurality of sensors comprising electrodes configured to detect electromyography (EMG) and positional signals, wherein the sensor strand is mechanically configurable to have a first shape during insertion and a second shape during operation, the second shape arranging the plurality of sensors in two or more rows; and an electronics suite comprising a housing, and a processor receiving signals from the plurality of sensors.

Example 2. The patient implantable system of the preceding example, wherein the sensor strand staggers, flexes, or bends in a plurality of directions.

Example 3. The patient implantable system of any of the preceding examples, further comprising a draw string of an adjustable length controlling the second shape of the sensor strand.

Example 4. The patient implantable system of any of the preceding examples, wherein the second shape defines a sensor array bracelet.

Example 5. The patient implantable system of any of the preceding examples, wherein the sensor array bracelet is mechanically configurable to have a variable radius.

Example 6. The patient implantable system of any of the preceding examples, wherein the sensor array bracelet comprises an open-end bracelet or a coiled bracelet.

Example 7. The patient implantable system of any of the preceding examples, wherein the sensor array bracelet comprises circumferentially distanced ends or longitudinally distanced ends.

Example 8. The patient implantable system of any of the preceding examples, wherein the electronic suite is coupled to a prosthetic device.

Example 9. The patient implantable system of any of the preceding examples, wherein the electronic suite comprises a non-rechargeable battery or a rechargeable battery.

Example 10. The patient implantable system of any of the preceding examples, wherein the rechargeable battery is coupled to a battery charging coil.

Example 11. The patient implantable system of any of the preceding examples, wherein the battery is hermetically or non-hermetically sealed.

Example 12. The patient implantable system of any of the preceding examples, wherein the electronic suite is attached to an end of the sensor strand or a middle portion of the sensor strand.

Example 13. The patient implantable system of any of the preceding examples, wherein the electronics suite is attached to a reference electrode.

Example 14. The patient implantable system of any of the preceding examples, wherein the electronic suite comprises an antenna for transmitting the signals received from the plurality of sensors.

Example 15. The patient implantable system of any of the preceding examples, wherein the housing of the electronics suite comprises a disc shape or a tubular shape with smooth edges.

Example 16. The patient implantable system of any of the preceding examples, comprising a plurality of feedthroughs transmitting the patient data from the sensory strand through the housing of the electronics suite by maintaining an hermeticity of the housing.

Example 17. The patient implantable system of any of the preceding examples, wherein the sensor strand comprises up to 32 electrodes.

Example 18. The patient implantable system of any of the preceding examples, wherein the sensor strand is formed of a flexible material coupled to a stylet shaping the sensor strand for insertion.

Example 19. A patient implantable system for subdermal collection of patient data, the patient implantable system comprising: a sensor strand comprising a plurality of sensors, wherein the sensor strand is mechanically configurable to have a first shape during insertion and a second shape during operation, the second shape arranging the plurality of sensors in two or more rows; and an electronics suite comprising a housing, and an electronic circuit disposed within the housing, the electronic circuit receiving signals from the plurality of sensors, the housing comprising a polymeric encapsulation layer surrounding the electronic circuit, providing a non-hermetic seal that protects the electronic circuit from bodily fluids.

Example 20. The patient implantable system of the preceding example, wherein the polymeric encapsulation layer comprises at least one of polyurethane, silicone, or polyethylene.

Example 21. The patient implantable system of any of the preceding examples, wherein the encapsulation layer provides environmental resistance during a temporary implantation, and provides controlled access to the electronic circuit for replacement or revision.

Example 22. The patient implantable system of any of the preceding examples, wherein the electronic circuit comprises a coating applied directly to the electronic circuit prior to encapsulation.

Example 23. The patient implantable system of any of the preceding examples, wherein the housing is flexible, conforming to anatomical structures during or after implantation.

Example 24. The patient implantable system of any of the preceding examples, wherein the sensors are configured for neuromodulation or cardiac monitoring.

Example 25. The patient implantable system of any of the preceding examples, wherein the polymeric encapsulation is applied using a dip-coating, spray-coating, or overmolding process.

Example 26. A method of using a patient implantable system for collecting internal patient data, the method comprising: inserting a stylet within a sensor strand comprising a plurality of sensors housed within a tubular body, the plurality of sensors comprising electrodes configured to detect electromyography (EMG) and positional signals, wherein the sensor strand has a first shape during insertion; positioning the sensor strand between tissue layers; and actuating a strand shape controller for the sensor strand to form and maintain a second shape during operation.

Example 27. The method of the preceding example, wherein the stylet is removed after forming the second shape.

Example 28. A modular insertion system comprising: a flexible conduit configured for implantation or subcutaneous positioning between tissue planes of a limb; and a removable stylet positioned within the conduit, the stylet having a stiffness that facilitates insertion of the conduit between anatomical tissue planes.

Example 29. The modular insertion system of any of the preceding examples, wherein the removable stylet is removed after placement of the flexible conduit, wherein the flexible conduit assumes a neutral or more conformable shape governed by a flexibility of a conduit material.

Example 30. The modular insertion system of any of the preceding examples, wherein the removable stylet is replaced with a second stylet comprising a greater flexibility than the removable stylet, the second stylet imparting a smooth curvature following contours of the limb.

Example 31. The modular insertion system of any of the preceding examples, wherein the removable stylet comprises a pre-formed curvature and a stiffness level adapted to conform to an anatomical structure and an appendage size.

Example 32. The modular insertion system of any of the preceding examples, wherein the removable stylet is composed of a shape-memory material.

Example 33. The modular insertion system of any of the preceding examples, wherein the shape-memory material is insertable in a substantially straight configuration.

Example 34. The modular insertion system of any of the preceding examples, wherein the shape-memory material in response to exposure to body temperature or physiological conditions, assumes a curved or coiled geometry to conform to a surrounding anatomical structure.

What is claimed is:

1. A patient implantable system for subdermal collection of patient data, the patient implantable system comprising:
a sensor strand comprising a plurality of sensors housed within a tubular body, the plurality of sensors comprising electrodes configured to detect electromyography (EMG) and positional signals, wherein the sensor strand is mechanically configurable to have a first shape during insertion and a second shape during operation, the second shape arranging the plurality of sensors in two or more rows; and
an electronics suite comprising a housing, and a processor receiving signals from the plurality of sensors.

2. The patient implantable system of claim 1, wherein the sensor strand staggers, flexes, or bends in a plurality of directions.

3. The patient implantable system of claim 1, further comprising a draw string of an adjustable length controlling the second shape of the sensor strand.

4. The patient implantable system of claim 1, wherein the second shape defines a sensor array bracelet, wherein the sensor array bracelet is mechanically configurable to have a variable radius.

5. The patient implantable system of claim 4, wherein the sensor array bracelet comprises an open-end bracelet or a coiled bracelet.

6. The patient implantable system of claim 4, wherein the sensor array bracelet comprises circumferentially distanced ends or longitudinally distanced ends.

7. The patient implantable system of claim 1, wherein the electronic suite is coupled to a prosthetic device.

8. The patient implantable system of claim 1, wherein the electronic suite comprises a non-rechargeable battery or a rechargeable battery.

9. The patient implantable system of claim 8, wherein the non-rechargeable battery or the rechargeable battery is hermetically or non-hermetically sealed.

10. The patient implantable system of claim 1, wherein the electronic suite is attached to an end of the sensor strand or a middle portion of the sensor strand.

11. The patient implantable system of claim 1, wherein the housing of the electronics suite comprises a disc shape or a tubular shape with smooth edges.

12. The patient implantable system of claim 1, comprising a plurality of feedthroughs transmitting the patient data from the sensory strand through the housing of the electronics suite by maintaining an hermeticity of the housing.

13. The patient implantable system of claim 1, wherein the sensor strand comprises up to 32 electrodes.

14. The patient implantable system of claim 1, wherein the sensor strand is formed of a flexible material coupled to a stylet shaping the sensor strand for insertion, wherein the stylet comprises an adjustable size, conforming to an anatomical structure and an appendage size.

15. A patient implantable system for subdermal collection of patient data, the patient implantable system comprising:
a sensor strand comprising a plurality of sensors, wherein the sensor strand is mechanically configurable to have a first shape during insertion and a second shape during operation, the second shape arranging the plurality of sensors in two or more rows; and
an electronics suite comprising a housing, and an electronic circuit disposed within the housing, the electronic circuit receiving signals from the plurality of sensors, the housing comprising a polymeric encapsulation layer surrounding the electronic circuit, providing a non-hermetic seal that protects the electronic circuit from bodily fluids.

16. The patient implantable system of claim 15, wherein the polymeric encapsulation layer comprises at least one of polyurethane, silicone, or polyethylene.

17. The patient implantable system of claim 15, wherein the encapsulation layer provides environmental resistance during a temporary implantation, and provides controlled access to the electronic circuit for replacement or revision.

18. The patient implantable system of claim 15, wherein the electronic circuit comprises a coating applied directly to the electronic circuit prior to encapsulation.

19. The patient implantable system of claim 15, wherein the housing is flexible, conforming to anatomical structures during or after implantation.

20. The patient implantable system of claim 15, wherein the sensors are configured for neuromodulation or cardiac monitoring.

21. The patient implantable system of claim 15, wherein the polymeric encapsulation is applied using a dip-coating, spray-coating, or overmolding process.

22. A method of using a patient implantable system for collecting internal patient data, the method comprising:
inserting a stylet within a sensor strand comprising a plurality of sensors housed within a tubular body, the plurality of sensors comprising electrodes configured to detect electromyography (EMG) and positional signals, wherein the sensor strand has a first shape during insertion;
positioning the sensor strand between tissue layers; and
actuating a strand shape controller for the sensor strand to form and maintain a second shape during operation.

23. The method of claim 22, wherein the stylet is removed after forming the second shape.

24. The method of claim 22, wherein the stylet comprises an adjustable size, conforming to an anatomical structure and an appendage size.
